(19) United States
(12) Patent Application Publication
(10) Pub. No.: US 2026/0224890 A1
(43) Pub. Date: Aug. 6, 2026

(54) RECONFIGURABLE MULTI-ELECTRODE APPARATUS

(71) Applicant: President and Fellows of Harvard College, Cambridge, MA (US)

(72) Inventors: Donhee Ham, Cambridge, MA (US); Jun Wang, Cambridge, MA (US); Yuchang Zhang, Cambridge, MA (US)

(73) Assignee: President and Fellows of Harvard College, Cambridge, MA (US)

(21) Appl. No.: 19/151,905

(22) PCT Filed: Jan. 26, 2024
(86) PCT No.: PCT/US2024/013023

§ 371 Date: Jul. 29, 2025

Related U.S. Application Data
(60) Provisional application No. 63/482,262, filed on Jan. 30, 2023.

(51) Int. Cl. A61N 1/05; A61N 1/36

(52) U.S. Cl. CPC A61N 1/0529; A61N 1/36125; A61N 1/36153; A61N 1/36157; A61N 1/36185

(57) ABSTRACT

Multi-electrode apparatuses and methods of using multi-electrode apparatuses are provided. The apparatuses each include a switching arrangement configured to selectively connect a group of one or more electrodes of the apparatus to a group of one or more circuits of the apparatus. Each electrode of the group may be selectively connected to multiple circuits each configured to provide a stimulation signal to the electrode concurrently, thus selectively increasing a range of amplitudes of an signal used to stimulate the electrode compared with a single circuit, and each configured to sense a signal from the electrode concurrently, thus permitting a signal-to-noise ratio (SNR) to be increased compared with a single circuit. The switching arrangement may be reconfigured to change a number of circuits connected to each electrode of the group, thus permitting the range to be further increased and the SNR to be further increased.

### CROSS REFERENCE TO RELATED APPLICATIONS

The present application claims the benefit of priority of U.S. Provisional Application No. 63/482,262 filed Jan. 30, 2023, entitled “RECONFIGURABLE MULTI-ELECTRODE APPARATUS,” the entire contents of which is incorporated by reference herein.

### FIELD

The present disclosure generally relates to apparatuses and methods that allow a plurality of electrodes and a plurality of circuits to be connected to each other selectively, such that an electrode may receive stimulation signals concurrently from multiple circuits, and such that multiple circuits may each sense a same signal concurrently from an electrode. The ability to address an electrode with two or more circuits may permit a wider range of signal amplitudes for stimulation of the electrode and also may permit a larger signal-to-noise ratio (SNR) of signals sensed from the electrode.

### BACKGROUND

Arrays of electrodes have become increasingly popular for a wide variety of applications, such as for electrochemical reactions to screen for biological activity and to prepare chemical libraries, and for electrophysiological stimulation of biological organisms (e.g., animal organs, plants, microorganisms, etc.) and sensing of responses from the organisms, to name a few. For example, multi-electrode arrays (MEAs) have been used for neurological tests to investigate biological responses to various experimental conditions, in vivo as well as in vitro.

In some cases, MEAs based on complementary metal-oxide-semiconductor (CMOS) technology have been found to be particularly suitable for electrical stimulation and sensing of biological organisms due to their ability to be fabricated on semiconductor chips using known microfabrication technologies, thus permitting MEAs to have a wide range of sizes, from dimensions in the submicron range and even in the nanometer range to dimensions that may be limited predominantly by dimensions of wafers from which the chips are cut. Using such technologies, MEAs may be produced to have a high degree of integration and to coexist with other on-chip circuitry, i.e., an MEA and circuitry for addressing electrodes of the MEA may be located on a single chip. A high degree of integration may be beneficial for some investigations where stimulation and/or sensing over very short distances is desirable. Having a MEA coexist with on-chip circuitry on a same semiconductor chip may be beneficial to reduce an amount of wiring between the electrodes and the on-chip circuitry, which may reduce an amount of noise that otherwise could be present if longer interconnection wiring were used.

### SUMMARY OF THE DISCLOSURE

The inventors have recognized and appreciated a number of considerations for MEAs. One consideration is that as a total number of electrodes of a MEA increases the complexity of input-output schemes for stimulating the electrodes and sensing responses from the electrodes may also increase. Another consideration is that sensed signals may be small and, in some cases, may have amplitudes similar to that of measurement noise (e.g., environmental noise, circuit noise, and/or other noise picked up during sensing). A further consideration is that densely spaced electrodes may be desirable for some types of measurements for an investigation but for other types of measurements for the same investigation it may be desirable to have sparsely spaced electrodes. Another consideration is that for some investigations it may be desirable to be able to stimulate the electrodes with a wide range of currents, such as currents spanning orders of magnitude, e.g., from picoamps (pA) to microamps (μA) to milliamps (mA).

Aspects of the technology disclosed herein relate to techniques for increasing the signal-to-noise ratio (SNR) of signals sensed from electrodes of a multi-electrode apparatus and for expanding a range of amplitudes of stimulation signals provided to the electrodes of the multi-electrode apparatus. The multi-electrode apparatus may include one or more arrays of electrodes totaling tens of electrodes or hundreds of electrodes or thousands of electrodes or millions of electrodes. The disclosed technology also may be used to perform fine sensing from electrodes that are spaced relatively densely, or to perform sparse sensing from electrodes that are spaced relatively sparsely, or to perform fine sensing concurrently with sparse sensing, using a single chip. Such flexibility may be advantageous for investigations involving in vivo testing of biological organisms.

According to an aspect of the technology of the present disclosure, a multi-electrode apparatus is provided. The apparatus may comprise: a plurality of electrodes; a plurality of circuits configured to provide signals to and/or sense signals from the electrodes; routing lines comprising switches, the routing lines being configured to selectively connect the electrodes to the circuits; and a control circuitry configured to control the switches such that at least one of the electrodes is connected concurrently to two or more of the circuits.

In some embodiments of this aspect, the control circuitry may be configured to control the switches such that the two or more of the circuits are connected concurrently to a single one of the electrodes.

In some embodiments of this aspect, the control circuitry may be configured to control the switches such that the two or more of the circuits are connected concurrently to at least two of the electrodes.

In some embodiments of this aspect, the control circuitry may be configured to control the switches such that a first group of one or more of the electrodes is connected concurrently to a first plurality of the circuits, and such that a second group of one or more one of the electrodes is connected concurrently to a second plurality of the circuits.

In some embodiments of this aspect, the control circuitry may be configured to receive configuration data from an external device and to store the configuration data in the configuration memory as configuration bits for configuring a state of each of the switches.

In some embodiments of this aspect, the circuits may be configured to perform any one or any combination of: sense a current, sense a voltage, output a stimulation current, and output a stimulation voltage.

In some embodiments of this aspect, the circuits may be analog circuits.

In some embodiments of this aspect, the circuits and at least some of the electrodes may be disposed on a single substrate. In some embodiments, the single substrate may be a semiconductor chip.

In some embodiments of this aspect, the electrodes may comprise at least one electrode array. In some embodiments, the at least one electrode array may include a first array of at least two electrodes disposed on the semiconductor chip.

In some embodiments of this aspect, the apparatus may further comprise a shank configured to be inserted in an object. The at least one electrode array may include a first array of at least two electrodes disposed on the semiconductor chip, and may include a second array of at least two electrodes disposed on the shank.

In some embodiments of this aspect, a total number of the circuits on the semiconductor chip may be less than a total number of the electrodes. In some embodiments, the control circuitry may be configured to set the switches in a first configuration in which a selected group of electrodes is connected to the circuits such that each electrode of the selected group is connected to at least two of the circuits. In some embodiments, the control circuitry may be configured to set the switches in a second configuration such that each electrode of the selected group is connected to at least four of the circuits. In some embodiments, the control circuitry may be configured to set the switches in the second configuration based on whether a signal-to-noise (SNR) ratio of a first averaged signal determined from signals sensed by the at least wo of the circuits is at or above a threshold.

In some embodiments of this aspect, for each of the at least one of the electrodes, the two or more of the circuits connected concurrently to the electrode may each be configured to sense a signal from the electrode and to provide an output signal to the controller. The control circuitry may be configured to receive the output signals from the two or more of the circuits, compute an average of the output signals, and record the average as a reading for the electrode.

In some embodiments of this aspect, for each of the at least one of the electrodes, the two or more of the circuits connected concurrently to the electrode may each be configured to provide a stimulation signal to the electrode concurrently. In some embodiments, the stimulation signals provided by the two or more of the circuits may have a same amplitude. In some embodiments, the stimulation signals provided by the two or more of the circuits may have different amplitudes. In some embodiments of this aspect, for each of the at least one of the electrodes, the two or more of the circuits connected concurrently to the electrode may each be configured to provide a stimulation signal to the electrode concurrently and to provide the stimulation signal to another electrode concurrently.

In some embodiments of this aspect, the apparatus may further comprise bus lines configured to connect the circuits to an off-chip device. A total number of the bus lines may be equal to or greater than a total number of the circuits. Each of the circuits may be connected to at least one of the bus lines. In some embodiments, the apparatus may further comprise a circuit board on which the chip is mounted. The circuit board may comprise back-end circuitry. The circuits may be front-end circuits, and may connect to the back-end circuitry via the bus lines. In some embodiments, the back-end circuitry may include a field-programmable gate array (FPGA), and the bus lines may connect the front-end circuits to the FPGA. In some embodiments, the back-end circuitry may include any one or any combination of: a power supply, an input/output (I/O) interface, a bias-voltage source, and a reference-voltage source.

In some embodiments of this aspect, the electrodes may be part of a microelectrode array (MEA) of electrodes and may be configured to perform in vitro sensing and/or stimulation of an object. In some embodiments, the in vitro sensing and/or stimulation may be extracellular. In some embodiments, the in vitro sensing and/or stimulation may be intracellular.

In some embodiments of this aspect, the electrodes may be part of a microelectrode array (MEA) of electrodes and may be configured to perform in vivo sensing and/or stimulation of an object. In some embodiments, the in vivo sensing and/or stimulation may be extracellular. In some embodiments, the in vivo sensing and/or stimulation may be intracellular. In some embodiments, the electrodes may be configured to perform local field potential (LFP) sensing of the object. In some embodiments of this aspect, the electrodes may be configured to perform open-circuit voltage (OCV) sensing of the object.

In some embodiments of this aspect, at least some of the electrodes may be disposed in a first area of the semiconductor chip, and at least some of the circuits may be disposed in second area of the semiconductor chip different from the first area. In some embodiments, the first area may be at least partially surrounded by the second area.

In some embodiments of this aspect, at least some of the electrodes may be disposed in a first area of the semiconductor chip, at least some of the circuits may be disposed in the first area of the chip, the electrodes in the first area may be located above or below the circuits in the first area in a stacked arrangement. In some embodiments, some of the electrodes may be disposed in an area of the semiconductor chip different from the first area. In some embodiments, some of the circuits may be disposed in an area of the semiconductor chip different from the first area.

According to another aspect of the technology of the present disclosure, a method of using a multi-electrode apparatus is provided. The method may comprise: controlling a plurality of switches in routing lines to selectively connect a plurality of electrodes to a plurality of circuits configured to provide signals to and/or sense signals from the electrodes. The controlling may control the switches such that at least one of the electrodes is connected concurrently to two or more of the circuits.

In some embodiments of this aspect, the controlling may control the switches such that the two or more circuits are connected concurrently to a single one of the electrodes.

In some embodiments of this aspect, the controlling may control the switches such that the two or more circuits are connected concurrently to at least two of the electrodes.

In some embodiments of this aspect, the controlling may control the switches such that a first group of one or more of the electrodes is connected concurrently to a first plurality of the circuits and a second group of one or more one of the electrodes is connected concurrently to a second plurality of the circuits.

In some embodiments of this aspect, the method may further comprise: receiving, from an external device, configuration data for configuring a state of each of the switches; and storing the configuration data in the configuration memory as configuration bits.

In some embodiments of this aspect, the circuits may be configured to perform any one or any combination of: sense a current, sense a voltage, output a stimulation current, and output a stimulation voltage.

In some embodiments of this aspect, the circuits may be analog circuits.

In some embodiments of this aspect, the electrodes and the circuits may be disposed on a single substrate. In some embodiments, the single substrate may be a single semiconductor chip. In some embodiments, the electrodes may comprise at least one electrode array. In some embodiments, a total number of the circuits on the semiconductor chip may be less than a total number of the electrodes on the semiconductor chip.

In some embodiments of this aspect, the controlling may set the switches in a first configuration in which a selected group of electrodes is connected to the circuits such that each electrode of the selected group is connected to a first number of the circuits. In some embodiments, based on whether a signal-to-noise (SNR) ratio of a first signal determined from signals sensed by the first number of the circuits is at or above a threshold, the controlling may set the switches in a second configuration in which the selected group of electrodes is connected to the circuits such that each electrode of the selected group is connected to a second number of the circuits, the second number being greater than the first number. In some embodiments, the first number of the circuits may be at least one of the circuits, and the second number of the circuits may be at least four of the circuits.

In some embodiments of this aspect, the method may further comprise, for each of the at least one of the electrodes, sensing, by the two or more of the circuits connected concurrently to the electrode, a signal from the electrode; and computing an average of the signals sensed by the two or more of the circuits and recording the average as a reading for the electrode. In some embodiments, the signals sensed by the two of more of the circuits may be local field potentials (LFPs). In some embodiments, the signals sensed by the two of more of the circuits may be open-circuit voltages (OCVs).

In some embodiments of this aspect, the method may further comprise, for each of the at least one of the electrodes, stimulating the electrode with two or more stimulation signals respectively provided by the two or more of the circuits connected concurrently to the electrode. In some embodiments, the two or more stimulation signals may be stimulation currents. In some embodiments, the two or more stimulation signals may have a same amplitude. In some embodiments, two or more stimulation signals may have different amplitudes. In some embodiments, the two or more stimulation signals may be provided to at least two of the electrodes concurrently.

### BRIEF DESCRIPTION OF THE DRAWINGS

A skilled artisan will understand that the accompanying drawings are for illustration purposes only. It also is to be understood that in some instances various aspects of the present disclosure may be shown exaggerated or enlarged to facilitate an understanding of the disclosure. In the drawings, like reference characters generally refer to like features, which may be functionally similar and/or structurally similar elements, throughout the various figures. The drawings are not necessarily to scale, as emphasis is instead placed on illustrating and teaching principles of the various aspects of the present technology. The drawings are not intended to limit the scope of the claims or the present disclosure in any way. In the drawings:

FIG. 1 shows a block diagram of a multi-electrode apparatus, according to some embodiments of the present technology;

FIG. 2A schematically depicts an arrangement of switches and routing lines for selectively connecting electrodes to front-end circuits, according to some embodiments of the present technology;

FIG. 2B schematically depicts an arrangement of electrodes and front-end circuits, in which some electrodes are located separately from the front-end circuits and some electrodes are co-located with the front-end circuits on a one-to-one basis, and in which a switching matrix may be used to configure and reconfigure connections between the front-end circuits and any of the electrodes and/or any group or groups of the electrodes, according to some embodiments of the present technology;

FIG. 2C schematically shows arrangements in which an electrode is co-located with a front-end circuit and connectable to one or more other front-end circuits, according to some embodiments of the present technology;

FIG. 2D schematically shows arrangements in which an electrode is located separately from front-end circuits to which the electrode is connectable, according to some embodiments of the present technology;

FIG. 3 shows traces of signal amplitude as a function of time for a test signal (ORIGINAL-S) applied to an electrode, four signals (S1, S2, S3, S4) corresponding to the applied test signal and sensed by four different AFE circuits, and an averaged signal (AVERAGE-S) obtained from averaging the four sensed signals;

FIG. 4 shows a trace (A) of noise density as a function of frequency for a signal sensed from an electrode by one of four AFE circuits, and an averaged trace (B) for signals sensed from the electrode by the four AFE circuits; and

FIG. 5 schematically shows a multi-electrode apparatus for in vivo measurements, according to some embodiments of the present technology.

### DETAILED DESCRIPTION

Aspects of the present technology disclosed herein relate to techniques for increasing the signal-to-noise ratio (SNR) of signals sensed from electrodes of a multi-electrode apparatus and/or for expanding a range of amplitudes of stimulation signals provided to the electrodes of the multi-electrode apparatus. The electrodes may be, for example, part of one or more arrays of electrodes in a MEA. In some implementations of the disclosed technology, the techniques described herein may be used to perform fine sensing with densely spaced electrodes as well as sparse sensing with widely spaced electrodes, using a single semiconductor chip on which is disposed at least some of the electrodes and front-end circuitry for selectively addressing the on-chip electrodes as well as off-chip electrodes via controlled, selective connection of groups of one or more of the electrodes to groups of two or more circuits of the front-end circuitry. In some embodiments of the present technology, a single apparatus may be operated in a sparse-sensing mode and also in a fine-sensing mode. The sparse-sensing mode may be more desirable when studying a large object, so that signals may be sensed and recorded over a wider area (e.g., to study signal propagation in a large biological neural network), whereas the fine-sensing mode may be more desirable when studying features requiring high spatial resolution (e.g., to study regions near individual neurons).

Some embodiments of the present technology may be described in relation to MEAs, which may be incorporated in electrochemical apparatuses. In some embodiments, the MEAs may be incorporated in test apparatuses for in vivo investigations and/or in vitro investigations. It should be understood, however, that the present technology is not limited to applications involving MEAs and electrochemical apparatuses, or MEAs and in vivo and/or in vitro test apparatuses, or MEAs in general. The technology disclosed herein may also be applied to apparatuses with multiple electrodes that are not arrange in an array, in which it may be desirable to controllably increase SNR in signals sensed from the electrodes, and/or to controllably expand a range of amplitudes of stimulation signals applied to the electrodes, and/or to have flexibility to perform fine sensing or sparse sensing or both fine and sparse sensing using the electrodes.

A MEA may include a large number of electrode sites (e.g., 1000 to 10,000; 10,000 to 100,000; 100,000 to 500,000; 500,000 to 1,000,000; over 1,000,000). The sites may be controlled individually, or collectively, or in groups of one or more sites. The sites may be sensed selectively, such that a signal may be detected or sensed from sites individually, i.e., one at a time, or a plurality of signals may be detected or sensed concurrently from groups of one or more sites. The sites may be stimulated selectively, such that a first group of one or more sites may be stimulated with a first signal, a second group of one or more sites may be stimulated with a second signal different from the first signal, a third group of one or more sites may be stimulated with a third signal different from the first and second signals, etc. The first, second, and third groups of sites may be stimulated concurrently or at different times (e.g., sequentially).

According to some embodiments of the present technology, a plurality of electrodes may be disposed on a semiconductor chip and may be fabricated using CMOS technology. The chip may also include electrical circuitry configured to interact with the electrodes. The chip may be comprised of a plurality of electrode sites, with each site including a single electrode or a group of two or more electrodes. In some embodiments, a site may include a stimulation electrode and a sensing electrode. In some embodiments, a site may include a single electrode together with a circuit configured to provide a stimulation signal to the electrode and to sense a signal from the electrode. In some embodiments, a site may include a circuit together with a stimulation electrode and a sensing electrode, with the circuit being configured to provide a stimulation signal the stimulation electrode and to sense a signal from the sensing electrode. In some embodiments, sensing and stimulation may occur concurrently at a site.

According to some embodiments of the present technology, the chip may include a plurality of electrode sites and a plurality of circuits configured to interact with the sites. In some embodiments, the circuits may be located at the sites (“co-located sites”) such that each site may include a corresponding one of the circuits. For example, a co-located site may include one or more electrodes overlaying a circuit. In some other embodiments, the circuits may be located on the chip separately from the sites. For example, the chip may include border or peripheral regions surrounding an interior region, with the sites being arranged in the interior region and with the circuits being arranged at one or more of the border or peripheral regions. In some other embodiments, the chip may include a plurality of co-located sites, a plurality of sites without corresponding circuits at the sites (“non-circuit sites”), and a plurality of circuits located separately from the non-co-located sites and separately from the non-circuit sites.

According to some embodiments of the present technology, a total number of circuits on a chip may be different from a total number of electrode sites on the chip. In some embodiments the total number of circuits may be less than the total number of electrode sites. In some embodiments, an electrode may occupy a relatively smaller area than a circuit on the chip. Therefore, a co-located site may occupy a relatively larger area than a non-circuit site on the chip. This relative difference in area may allow some non-circuit sites to be arranged relatively closer to each other than co-located sites. In some embodiments, the chip may include a region of non-circuit sites of finely spaced electrodes arranged in columns and rows at a center-to-center pitch (from site to site) in a range of about 8 μm to about 12 μm (e.g., about 10 μm). Such a fine spacing may allow, e.g., fine sensing of signals to be detected from a biological organism (e.g., a plant, an animal, a microorganism), such that changes may be observed at spacings of about, e.g., 10 μm. Such a fine spacing also may allow, e.g., stimulation signals to be varied over short distances of about, e.g., 10 μm. In some embodiments, the chip may include at least one region of co-located sites and/or at least one region of widely spaced non-circuit sites arranged in columns and rows at a center-to-center pitch (from electrode to electrode) in a range of about 50 μm to about 300 μm. Such a wide spacing may allow electrodes to be more readily bonded to other devices for, e.g., off-chip delivery of stimulation signals to the electrodes (or off-chip sensing of the electrodes). Bonding to the widely spaced electrodes may be via wire bonding, or solder-bump flip-chip bonding, or other known techniques. For example, externally provided stimulation signals may be delivered to electrodes of co-located sites through bond wires while corresponding circuits of the co-located sites and/or other circuits on the chip perform sensing of the electrodes. In another example, externally provided stimulation signals may be delivered to widely spaced electrodes through solder bumps attaching the electrodes to external circuitry while on-chip circuits connected to the electrodes sense signals from the electrodes being stimulated. It should be understood that circuits or circuit components that are “on” the chip may have some or all portions positioned on a surface of the chip and/or some or all portions positioned above the surface of the chip and/or some or all portions positioned below the surface of the chip. Similarly, it should be understood that electrodes that are “on” the chip may be partially or completely positioned on a surface of the chip, partially or completely positioned above the surface of the chip, and/or partially or completely positioned below the surface of the chip.

According to some embodiments of the present technology, electrodes may be electrically connected to circuits on the chip selectively and individually via on-chip routing buses, routing lines, and switches disposed in the routing lines. The switches may be controlled to open and close to permit individual electrodes on the chip or electrically connected to the chip to interact with one or more of the circuits on the chip and/or to permit individual circuits on the chip to interact with one or electrodes. As will be appreciated, for co-located sites, the circuit and the electrode(s) of the sites may be in a stacked arrangement and may be connected via one or more vertical routing lines (e.g., wiring extending perpendicular to a main surface of the chip). In some embodiments, the routing lines may include routing buses configured to transmit signals to a plurality of electrodes and/or a plurality of circuits.

According to some embodiments of the present technology, the chip may include an array of pixels, which may be arranged in columns and rows. Each pixel may include a group of one or more electrodes and a pixel circuit configured to control interactions with the electrode(s) of the pixel. In some embodiments, the pixel circuit may be comprised of an analog front-end circuit configured to be addressed by off-chip circuitry, such that the electrode(s) of the pixel may be stimulated according to a predetermined parameter provided by the off-chip circuitry via the pixel circuit. For example, the predetermined parameter may be a predetermined voltage provided to the pixel circuit of the pixel, which may determine an amplitude and polarity of a stimulation signal provided to the electrode(s). In some embodiments, each pixel of the array may be configured individually by signals provided by the off-chip circuitry, as discussed herein, such that localized stimulation may be performed at one or more selected pixels differently from stimulation performed at other pixels of the array and/or localized sensing may be performed at one or more selected pixels differently from sensing performed at other pixels of the array. In some embodiments, the off-chip circuitry may communicate with the pixel circuits via on-chip circuitry configured to transmit signals to/from the off-chip circuitry via, e.g., wires bonded to input-output pads on the chip. In some embodiments, the chip may be mounted on a printed-circuit board (“PCB”) on which is mounted the off-chip circuitry. The off-chip circuitry may, in turn, be controlled by signals from an external device such as, e.g., a host computer. In some embodiments, the co-located-sites discussed above may be comprised of an array of pixels.

The inventors have recognized and appreciated a need for techniques that permit spatially organized stimulation of an object (e.g., a biological organism, a collection of reaction cells, etc.) with electrical stimulation signals applied to individual sites in an area of the object and/or that permit spatially organized detection of electrical response signals from individual sites in an area of the object. For some applications, such as where an optimal value for a stimulation signal is not known, it may be desirable to be able to test a wide range of amplitudes for the stimulation signal. For example, some electrochemical reactions and some synthesis reactions may require application of an unknown amount of current or an unknown amount of voltage to initiate the reactions and therefore it would be beneficial to provide an apparatus that is able to individually stimulate a plurality of electrodes at different levels of current or voltage (e.g., a first group of electrodes at 1 pA, a second group of electrodes at 1 nA, a third group of electrodes at 1 mA, a and fourth group of electrodes at 1 A, etc.) For some applications, such as where response signals may be small and/or where it would be meaningful to detect slight variations in signal values, it may be desirable to provide a highly sensitive sensing scheme where small signals may be discerned even amongst background noise detected by the sensing scheme. The inventors have recognized and appreciated that, in some cases, it may be desirable for the response signals to be detected during and/or after the stimulation signals are applied, and to permit a localized response to be recorded for each individual site, e.g., to have the response sensed at the location where the stimulation signal is applied and to record the sensed response and/or a value based on the sensed response. It has also been recognized and appreciated by the inventors that, in some cases, it may be desirable for the response signals to be detected during and/or after the object is exposed to other types of stimuli (e.g., a change in the object's chemical environment).

The inventors have developed technology, disclosed herein, that permits electrodes to be individually stimulated with a wide range of amplitudes spanning orders of magnitude and that also permits sensitive electrical signals to be detected through a controllable increase in SNR. Typical conventional schemes do not provide such flexibility in a single apparatus. For example, small signals such as electrical signals indicative of neurological activity may be detected readily and discerned from noise detected with the signals, and such signals may be detected during stimulation with, e.g., currents range from picoamps to nanoamps to milliamps to amps. Apparatuses and devices employing the disclosed technology may be fabricated using known semiconductor fabrications techniques. Although embodiments of the present technology may be described herein for MEAs, aspects of the disclosed technology may be used generally in technology areas where it is desirable for multiple electrodes to be stimulated with signals spanning a wide range of amplitudes and where noise reduction or an enhancement of the SNR of sensed signals is desired.

As noted above, MEAs based on CMOS technology have become a widely used platform for investigations where electrodes are to receive electrical stimulation and/or where signals are to be detected (e.g., sensed and/or recorded) from the electrodes. MEA systems may include arrays of active electrode sites configured to generate local reactions that occur at electrodes of each of the sites (e.g., at electrode surfaces) when a voltage or a current is applied to the electrodes and/or to sense a local electrical condition at each of the sites (e.g., before and/or during and/or after stimulation). In some cases, MEA systems may use a voltage stimulation scheme because a stimulation voltage can be easily shared amongst electrodes of multiple sites and selectively applied via an on-chip switch network. In some cases, such as cases where a high degree of quantitative control over the stimulation signal is desired, a current stimulation scheme may be preferred over voltage stimulation because an amount of charge may be more readily quantified via control of an amount of current flow. As noted above, an electrode site may comprise a single electrode or may comprise a plurality of electrodes. For example, an electrode site may comprise a sensing electrode and a stimulation electrode.

Switching techniques are disclosed herein that may be used to expand a range of amplitudes of stimulation signals applied to electrode sites formed on a semiconductor chip, by controllably configuring opened or closed states of switches formed on the chip, thus allowing connection configurations between electrode sites and on-chip circuits to be changed or reconfigured. Also disclosed herein are switching techniques that may be used to increase SNRs of signals sensed at the sites. In some embodiments, the sites may be arranged as an array of pixels, with each pixel comprising an electrode site. In some embodiments, some or all of the pixels may each include a pixel circuit configured to address the electrode(s) corresponding to the pixel. In some embodiments, the array of pixels may comprise an array of electrode sites located adjacent an array of pixel circuits. For example, the array of electrode sites may be located above the array of pixel circuits such that a minimal amount of wiring is needed to connect an electrode site to a pixel circuit underneath the electrode site.

According to some embodiments of the disclosed technology, some or all of the electrode sites may be addressed by on-chip circuits that are selectively connectable to the electrode sites, to stimulate electrodes of the electrode sites and/or to sense signals from electrodes of the electrode sites. In some embodiments, one or more of the sites may each be connected selectively to a plurality of on-chip circuits, such that the sites may each be provided with a stimulation signal from the connected on-chip circuits concurrently. For example, an electrode of a site may be connected selectively to two on-chip circuits each configured to provide a stimulation current to the electrode site, thus advantageously allowing the electrode to be stimulated by a signal having an amplitude that is a sum of the stimulation currents of the two on-chip circuits. The two on-chip circuits may be connected selectively to the electrode by controlling a network of switches interconnecting the electrodes of the sites to the on-chip circuits, as discussed herein. In another example, an electrode of a site may be connected selectively to four on-chip circuits each configured to detect or sense a same current or a same voltage at the electrode, concurrently. The four signals sensed by the on-chip circuits may be provided to a circuitry configured to combine the signals to yield an overall sensed signal for the site, with the overall sensed signal having a higher SNR than a sensed signal detected by only one of the four on-chip circuit. The ability to increase the SNR by increasing the number of on-chip circuits connected to an electrode may be particularly advantageous when a signal sensed by a single on-chip circuit is found to be weak and difficult to discern from noise sensed together with the signal. In some embodiments, the flexibility to reconfigure a sensing arrangement for an electrode may allow the electrode to connect to an increasing number of on-chip circuits, which may advantageously permit the SNR to be increased until the overall sensed signal is discernible from the noise with a reasonable degree of confidence. In some embodiments, the on-chip circuits may be configured to sense open-circuit voltages (OCVs) at the sites. In some embodiments, an OCV of a site may be sensed during stimulation of an electrode of the site.

As noted herein, CMOS-based MEAs have been used for neurological investigations to investigate biological responses to various in vivo and in vitro experimental conditions. As will be appreciated, biological investigation sites may have a range of different size scales, and different biological activities may occur at distances that can be dense or relatively close to each other or that can be sparse or relatively far from each other. For example, a biological investigation site may have a diameter ranging from about a few centimeters (or greater) to a few millimeters (or smaller), and may have different biological reactions occurring at, e.g., sub-millimeter distances from each other (or even smaller).

Therefore, for some biological investigations, a MEA having a high degree of spatial resolution over a relatively large area may be desirable.

The inventors hare recognized and appreciated that CMOS-based MEAs may be able to provide an array of hundreds of thousands of electrodes (or greater) over a chip area of a few centimeters or greater; however, the chip area typically is shared by the electrodes and on-chip circuitry for stimulating the electrodes and reading out sensed signals from the electrodes, which can limit the area available for the electrodes. The inventors have recognized a need to maximize the number of electrodes in the chip area while having flexibility to address electrodes individually or collectively in groups of two or more, and also while being able to increase SNR of detected signals and/or increase a range of amplitudes of stimulation signals. Existing schemes that have been used in MEAs to maximize the number of electrodes typically provide a series of switches configured to allow one or more an on-chip circuits to each address electrodes individually, one by one. For example, for an array of n rows of electrodes, each row may be provided with an on-chip circuit configured to address electrodes of a corresponding row, such that n on-chip circuits may be provided on the chip for the n rows of the array. Such schemes allow a relatively small number of larger on-chip circuits to be used with a relatively large number of smaller electrodes on the same chip. However, the inventors have recognized and appreciated that such schemes do not provide flexibility to increase the range of amplitudes of stimulation signals nor do such schemes provide flexibility to increase the SNR for signals that are small (e.g., of the same amplitude as sensed noise). The inventors have further recognized and appreciated that, as the density of electrodes increases in the same amount of space on a chip, noise in signals sensed from the electrodes may increase. The inventors have recognized that in applications where signal noise may be problematic, such as when signals are to be sensed from closely spaced electrodes, the ability to bring about an increase in the SNR may be more important than in applications where signal noise is not of concern, such as when signals are to be sensed from electrodes that are relatively distant from each other. For example, neurological investigations of a local field potential or a sub-threshold neural membrane potential may use a MEA for extracellular sensing (e.g., sensing from sparsely situated electrodes) where noise in the sensed signals may not be problematic. In such investigations, subthreshold dynamics of single neurons are not being observed and therefore the ability to increase the SNR may not be important. On the other hand, the inventors have recognized that in applications where noise can be problematic, such as when detecting signals from individual (single) neurons, the flexibility to increase the SNR may be beneficial and, in some cases, critical to being able to discern a neuron-related signal from noise.

The inventors have developed a reconfigurable multi-electrode apparatus that provides flexibility to perform large-scale sensing of signals from densely situated electrodes on a semiconductor chip via on-chip circuits. The apparatus may be configured or reconfigured to increase a SNR of a signal sensed from an electrode by controlling at least one on-chip switching matrix of the apparatus, to increase a number of the on-chip circuits able to sense a signal from the electrode at same time. The apparatus also may be configured or to reconfigured to increase a range of amplitudes of a stimulation signal applied to one or more of the electrodes by controlling at switching matrix of the apparatus, to increase a number of the on-chip circuits able to output a stimulation signal to the one or more electrodes at the same time. In some embodiments of the present technology, the on-chip circuits may comprise part of “front-end” circuitry of the apparatus. In some embodiments, the on-chip circuits may be analog circuits and may be configured to receive and output analog signals. In some embodiments, front-end circuitry may include digital circuits configured to receive and output digital signals. In some embodiments, the front-end circuitry may include circuits able to receive analog and/or digital signals and output analog and/or digital signals. The on-chip circuits may be selectively connected to the electrodes via a routing system comprised of routing lines, buses, and switches. In some embodiments, off-chip circuitry may control opening and closing of the switches. The off-chip circuitry may be, e.g., circuitry disposed on a PCB and may include analog circuitry and/or digital circuitry and/or a combination of analog circuitry and digital circuitry. The off-chip circuitry may form at least part of “back-end” circuitry of the apparatus. Such flexibility to reconfigure how the electrodes are connected to the on-chip circuits may permit the apparatus to be used for a variety of different applications.

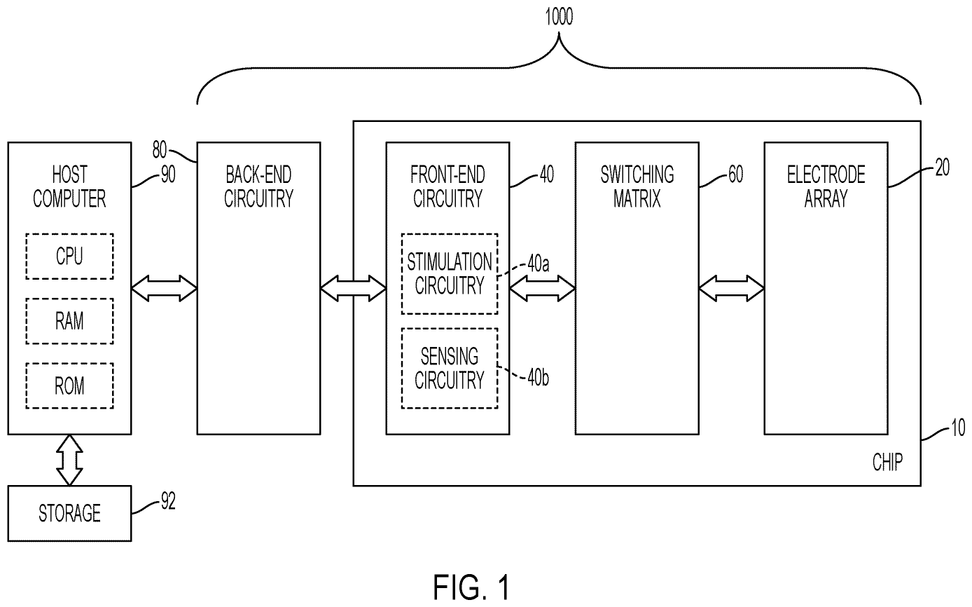

FIG. 1 schematically shows a multi-electrode apparatus 1000, according to some embodiments of the present technology. The apparatus 1000 may include an electrode array 20 in communication with and operably connected to front-end circuitry 40 via a switching matrix 60. In some embodiments, CMOS technology may be used to fabricate the electrode array 20, the front-end circuitry 40, and the switching matrix 60 on a single semiconductor chip 10. The front-end circuitry 40 may include any one or any combination of: electrodes, electrode-stimulation circuit(s), electrode-sensing circuit(s), amplifier(s), buffer(s), shift register(s), multiplexer(s), memory(ies), clock(s), switch(es), heater(s), temperature sensor(s), analog-to-digital converter(s) (ADC(s)), and the like. In some embodiments, the electrode array 20 may include off-chip electrodes in communication with and operably connected to the switching matrix 60 and/or the front-end circuitry 40 via wire bonding or other connection techniques. For example, the off-chip electrodes may be disposed on an implantable substrate configured for in vivo investigations and may be in electrical communication with the switching matrix 60 via wires connecting contact pads on the chip 10 with contact pads on the implantable substrate.

According to some embodiments of the present technology, the apparatus 1000 also may include back-end circuitry 80 in communication with and operably connected to the front-end circuitry 40. The back-end circuitry 80 may be located off-chip and may be connected to the front-end circuitry 40 via, e.g., wire bonds. In some embodiments, contact pads on the chip 10 may be connected with contact pads on a substrate (e.g., a PCB) on which the back-end circuitry 80 is disposed. In some embodiments, the back-end circuitry 80 may include any one or any combination of: voltage source(s) (e.g., reference voltage source(s), bias voltage source(s), and the like), power supply(ies), FPGA(s), digital-to-analog converter(s) (DAC(s)), ADC(s), input-output interface(s) (e.g., a USB interface), and the like. In some embodiments, the back-end circuitry 80 may be disposed on a PCB to which the chip 10 is mounted. As will be appreciated, other connection techniques may be employed for connecting the chip 10 to the back-end circuitry 80. In some embodiments, a host computer 90 may be in communication with and operably connected to the back-end circuitry 80 and may be configured to communicate signals to and receive signals from the front-end circuitry 40 and the switching matrix 60 via the back-end circuitry 80. In some embodiments, the host computer 90 may include at least one microprocessor (e.g., CPU(s)), volatile memory (e.g., RAM), and non-volatile memory (e.g., ROM). A storage device 92 may be operably connected to the host computer 90 and may be configured to record sensed data based on signals sensed from the array 20 and/or to store configuration data for controlling opened and closed states of switches of the switching matrix 60 and/or to store data for controlling stimulation data for configuring the front-end circuitry 40 to stimulate groups of electrodes of the array 20. For example, the host computer 90 may provide configuration data to the front-end circuitry 40 to update configuration bits stored in a configuration memory of the front-end circuitry 40. The configuration bits may be used by the apparatus 1000 to control a stimulation procedure or a sensing procedure. The storage device 92 may store computer-readable code executed by the microprocessor(s) to control one or more operations or procedures of the apparatus 1000. For example, the microprocessor(s) may execute the computer-readable code to control an stimulation procedure to stimulate the electrode array 20 and/or to control a sensing procedure to sense signals from the electrode array 20.

More specifically, according to some embodiments of the present technology, the front-end circuitry 40 may include circuits configured to stimulate the electrodes of the array 20 and/or to sense signals from the electrodes of the array 20. Some or all of these circuits may be analog circuits and may be referred to herein as analog front-end (AFE) circuits. In some embodiments, the AFE circuits of the front-end circuitry 40 may include stimulation circuitry 40a and sensing circuitry 40b. The stimulation circuitry 40a may be configured to provide electrical input signals (e.g., a current or a voltage) to cause stimulation of one or more electrodes of the array 20. Although the stimulation circuitry 40a is depicted in FIG. 1 to be a discrete unit of the front-end circuitry 40, it should be understood that the stimulation circuitry 40a may be comprised of a plurality of circuits located at a plurality of locations on the chip 10. The sensing circuitry 40b may be configured to detect or sense electrical signals from one or more electrodes of the array 20. Although the sensing circuitry 40b is depicted in FIG. 1 to be a discrete unit of the front-end circuitry 40, it should be understood that the sensing circuitry 40b may be comprised of a plurality of circuits located at a plurality of locations on the chip 10. In some embodiments, each AFE circuit may include a stimulation circuit of the stimulation circuitry 40a and a sensing circuit of the sensing circuitry 40b, and each AFE circuit may be controlled to output a stimulation signal to one or more of the electrodes of the array 20 and/or to sense a signal from at least one electrode of the array 20. In some embodiments, some or all of the AFE circuits may be associated with corresponding electrodes of the array 20 at co-located sites. For example, the AFE circuits may be the pixel circuits discussed herein. In some embodiments, some or all of the AFE circuits may be located on the chip 10 separately from the electrodes of the array 20. It should be understood that the electrodes of the array 20 need not be spatially arranged in a single regular pattern of regular columns or rows but instead may have any arrangement. In some embodiments, the front-end circuitry 40 may include the AFE circuits as well as additional on-chip circuitry configured to stimulate a selected electrode of the array 20 via the switching matrix 60 and/or to sense an OCV and/or a current flow at the selected electrode. In some embodiments, the OCV and/or the current flow may be sensed during stimulation of the selected electrode.

Additional details regarding electrode structures, multi-electrode arrays, pixels circuits, and stimulation and sensing of electrodes in a multi-electrode array may be found in International Patent Application No. PCT/US2022/033228 (Attorney Docket No. H0498.70743WO00), which is incorporated by reference herein in its entirety. In some embodiments, portions of the AFE circuits may be structured similar to the pixels circuits described in International Patent Application No. PCT/US2022/033228.

According to some embodiments of the present technology, the switching matrix 60 may selectively interconnect the electrodes of the array 20 with the AFE circuits of the front-end circuitry 40 via a plurality of routing lines and a plurality of switches positioned along the routing lines to enable configuration and reconfiguration of various electrodes for stimulation and/or for sensing. For example, the switches may be controlled to permit a first group of one or more electrodes of the array 20 to be stimulated by a first group of AFE circuits of the front-end circuitry 40 in a first stimulation configuration, and the switches may be controlled to permit a second group of one or more electrodes of the array 20 to be stimulated by the first group of AFE circuits or by a second group of AFE circuits of the front-end circuitry 40 in a second stimulation configuration. In another example, the switches may be controlled to permit one of the electrodes of the array 20 to be sensed by a first plurality of AFE circuits of the front-end circuitry 40 in a first sensing configuration, and the switches may then be controlled to permit the same electrode to be sensed by a second plurality of AFE circuits of the front-end circuitry 40 in a second sensing configuration. As will be appreciated, numerous types of stimulation configurations and sensing configurations may be achieved by appropriately controlling an opened or closed state of each of the switches of the switching matrix 60.

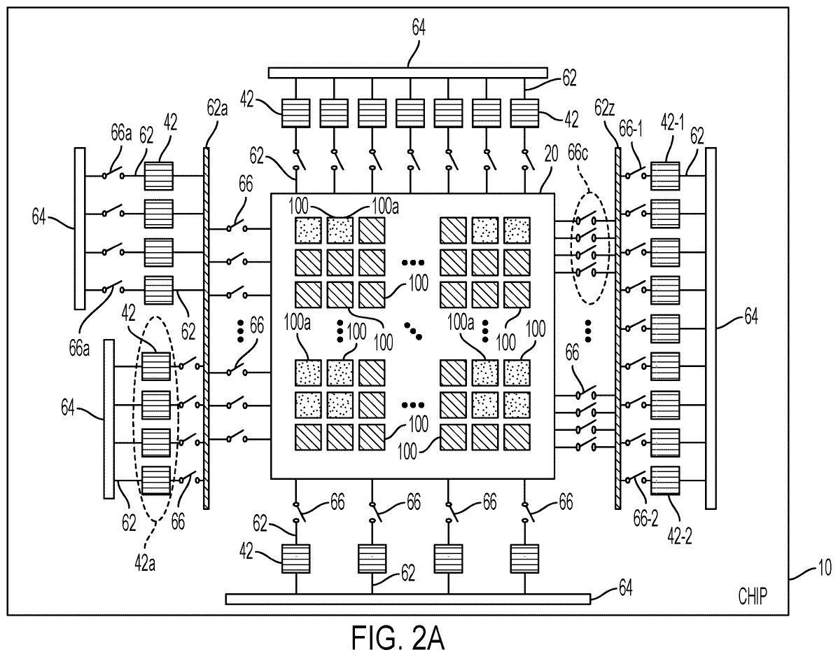

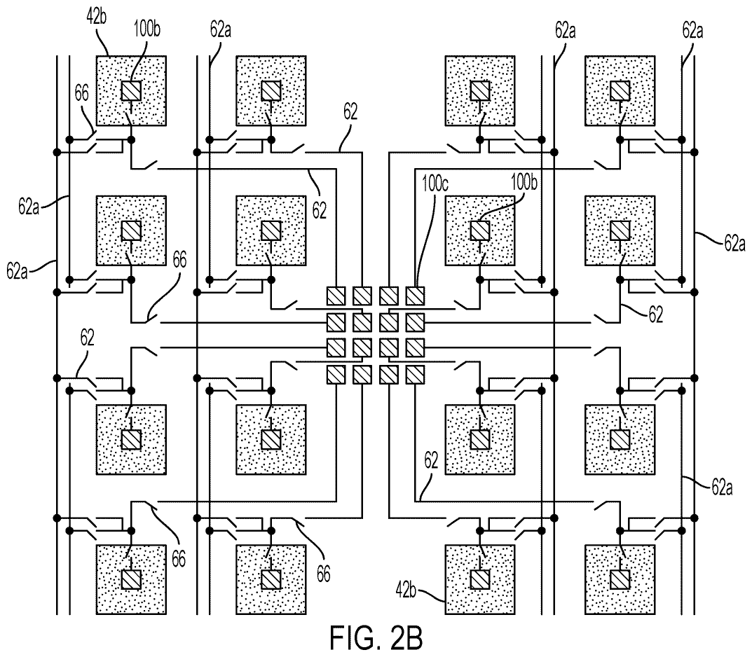

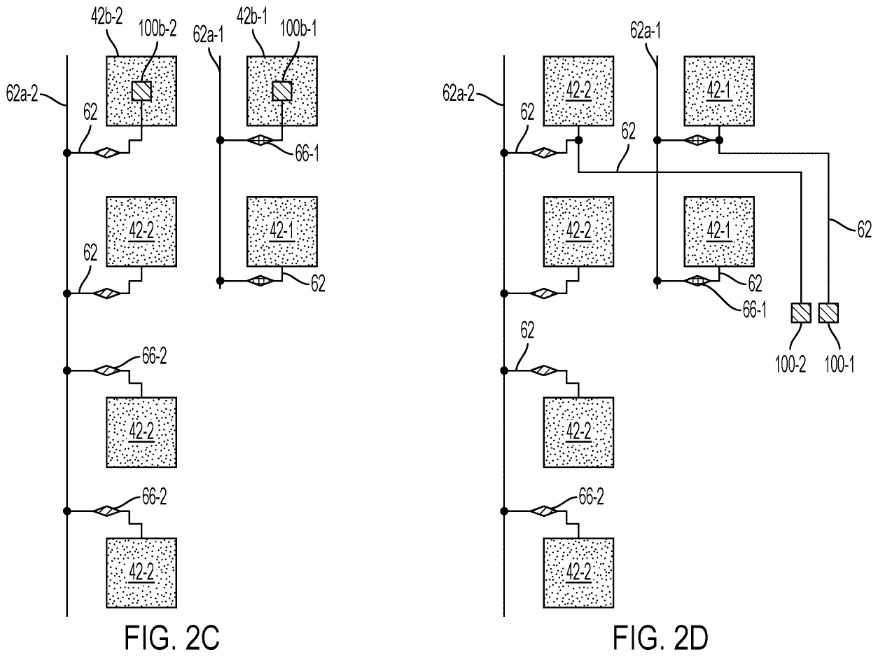

FIG. 2A schematically shows examples of connection configurations for connecting a plurality of electrodes 100 of the array 20 to a plurality of AFE circuits 42 of the front-end circuitry 40, according to some embodiments of the present technology. It should be understood that the apparatus 1000 may include any one or any combination of the connection configurations shown in FIG. 2A and/or one or more connection configurations not specifically shown in FIG. 2A.

In some embodiments of the present technology, the AFE circuits 42 may be configured to provide stimulation signals to the electrodes 100 and sense signals from the electrodes 100 via routing lines 62, buses 64, and switches 66. In some embodiments, the buses 64 may transmit signals to and from the AFE circuits 42 via the switches 66 and the routing lines 62. In some embodiments, the buses 64 may transmit signals to and from the AFE circuits 42 directly via the routing lines 62, without an intervening switch. For example, a control signal from the back-end circuitry 80 may be provided directly to a group 42a of the AFE circuits 42 via the routing lines 62 without intervening switches. In some embodiments, the routing lines 62 may include routing buses 62a, 62z configured to permit one or another of the AFE circuits 42, or a plurality of the AFE circuits 42, to communicate with a plurality of the electrodes 100. In a first example, a single circuit 42-1 of the AFE circuits 42 may output a stimulation signal to a plurality of selected electrodes 100a of the electrodes 100 of the array 20 via the routing bus 62z, a switch 66-1 between the circuit 42-1 and the routing bus 62z, and a group 66c of switches 66 between the routing bus 62z and the selected electrodes 100a. The selected electrodes 100a are schematically depicted to be relatively lighter in color in FIG. 2A, in comparison with non-selected ones of the electrodes 100. In a second example, the circuit 42-1 and another circuit 42-2 of the AFE circuits 42 may each output a stimulation signal to the selected electrodes 100a via switches 66-1, 66-2, the routing bus 62z, and the group 66c of switches 66 between the routing bus 62z and the selected electrodes 100a. For instance, the circuit 42-1 may provide up to a maximum current of x ma to each of the selected electrodes 100a, and the circuit 42-2 may provide up to a maximum current of y pa to the each of selected electrodes 100a, such that each of the electrodes 100a may be stimulated with up to a maximum current of (x ma+y pa), which is greater than an amount of current capable of being provided by the circuit 42-1 or the circuit 42-2 alone. In some embodiments, the circuits 42-1, 42-2 may be configured to output the same amount of current. In FIG. 2A, the circuits 42-1, 42-2 are shown to be connected to the same bus 64 but in some embodiments they may be connected to different buses 64.

In FIG. 2A, the electrodes 100 and the AFE circuits 42 are not co-located on the chip 10 but instead are located in separate areas of the chip 10. This may allow the electrodes 100 to be more closely spaced relative to each other, which may allow a greater total number of electrodes 100 to be present on the chip 10. According to some embodiments of the present technology, the AFE circuits 42 may be arranged to surround the electrodes 100. For example, the chip 10 may have a square shape, the AFE circuits 42 may be arranged near a periphery of the chip 10, and the electrodes 100 may be arranged in a region surrounded by the AFE circuits 42, such as schematically shown in FIG. 2A. In some embodiments, the AFE circuits 42 may be arranged in one or more rows near one or more peripheral sides of the chip 10.

FIG. 2B schematically shows an arrangement in which some electrodes 100b of the array 20 are co-located with some circuits 42b of the AFE circuits 42 of the front-end circuitry 40 on a one-to-one basis, according to some embodiments of the present technology. For example, the electrodes 100b may overlay their corresponding circuits 42b and may be electrically connected to their corresponding circuits 42b by vertically oriented conductive lines formed by known CMOS microfabrication techniques. In some embodiments, each of the circuits 42b may be configured to provide a stimulation signal to, and/or to sense a signal from, a corresponding one of the electrodes 100b overlaying the circuit 42b without requiring an intervening one of the switches 66 to be activated (i.e., closed). A beneficial aspect of having the electrodes 100b overlaying the circuits 42b is that minimal wiring and/or other interconnection components (e.g., switches) are needed for transmission of signals between the circuits 42b and their corresponding electrodes 100b. A reduction in wiring may, in some cases, reduce signal noise, which may be particularly important for sensing low-amplitude signals. In some embodiments, the array 20 also may include some non-overlaying electrodes 100c that do not overlay any of the circuits 42b and that are connectable to the circuits 42b via routing lines 62, buses 42a, and switches 66, as depicted in FIG. 2B. As noted herein, a beneficial aspect of having the electrodes 100c arranged separately from the circuits 42b is that a higher density of the electrodes 100c per unit area may be possible relative to the electrodes 100b overlaying the circuits 42b due to the larger size requirements of each of the circuits 42b. The presence of the closely spaced electrodes 100c may permit, e.g., evaluation of chemical and/or biological reactions over a finer distance scale and/or may permit, e.g., stimulation of chemical and/or biological structures that are closely spaced relatively to each other. In some embodiments, a plurality of co-located electrodes 100b may overlay a single co-located circuit 42b (not shown) and may be configured to be sensed by and/or stimulated by that overlayed circuit 42b and/or by another AFE circuit 42 on the chip 10 (not shown).

More specifically, according to some embodiments of the present technology, the routing lines and buses 62, 62a and the switches 66 along the routing lines 62 may interconnect the circuits 42b and the electrodes 100b, 100c such that a connection may be established between any one or more of the circuits 42b and any one or more of the electrodes 100b, 100c through appropriately controlling a state (opened or closed) of the switches 66. For example, in FIG. 2B, the switches 66 may be controlled such that one or more of the circuits 42b may each be connected to an additional one or more of the non-overlaying electrodes 100c concurrently with a connection to a co-located one of the electrodes 100b. The electrodes 100b that are co-located with the circuits 42b may be relatively sparsely arranged, as schematically depicted in FIG. 2B, and may be referred to as “sparse” electrodes 100b, and the electrodes 100c that are not co-located may be referred to as “dense” electrodes 100c. In some embodiments, a distance separating the sparse electrodes 100b may be sufficient to permit the electrodes 100b to be electrically connected to external or off-chip circuitry via, e.g., wire bonding techniques and/or flip-chip bonding techniques. Although not shown in FIG. 2B, the co-located electrodes 100b also may be connectable to non-co-located AFE circuits 42 on the chip 10 via the routing lines 62, buses 42a, and switches 66. The sparse electrodes 100b may each be stimulated respectively via their co-located circuits 42b and in addition may be stimulated, concurrently or at separate times, by another one or more of the AFE circuits 42, 42b on the chip 10 and/or by off-chip circuitry. Similarly, the sparse electrodes 100b may each be sensed respectively via their co-located circuits 42b and in addition may be sensed, concurrently or at separate times, by another one or more of the AFE circuits 42, 42b on the chip 10 and/or by off-chip circuitry. In short, by appropriately setting the open or closed states of the switches 66, each electrode 100 of the apparatus 1000 may be connected to a single AFE circuit 42 or a plurality of AFE circuits 42 of the apparatus 1000 concurrently, and, similarly, each AFE circuit 42 of the apparatus 1000 may be connected to a single electrode 100 of the apparatus or a plurality of electrodes 100 of the apparatus concurrently.

FIG. 2C schematically shows arrangements in which sparse electrodes 100b-1, 100b-2 are co-located with AFE circuits 42b-1, 42b-2, respectively. The electrode 100b-1 is co-located with and electrically connected to the AFE circuit 42b-1 and also is connected to another AFE circuit 42-1 via a routing bus 62a-1 and routing lines 62. In FIG. 2C, switches 66-1 in the routing lines 62 connecting the electrode 100b-1 to the AFE circuits 42b-1, 42-1 are in the closed state. The electrode 100b-1 may therefore be stimulated by stimulation signals provided concurrently by the two AFE circuits 42b-1, 42-1, which may allow the electrode 100b-1 to be stimulated by a higher-amplitude signal than possible by stimulation of the co-located AFE circuit 42b-1 alone. The two AFE circuits 42b-1, 42-1 may provide signals of different amplitudes to the electrode 100b-1 or signals of a same amplitude.

Similarly, the electrode 100b-2 is co-located with and electrically connected to the AFE circuit 42b-2 and also is connected to three other AFE circuits 42-2 via a routing bus 62a-2 and routing lines 62. Switches 66-2 in the routing lines 62 connecting the electrode 100b-2 and the AFE circuits 42b-2, 42-2 are in the closed state. The electrode 100b-2 may therefore be stimulated by stimulation signals provided concurrently by the four AFE circuits 42b-2, 42-2, which may allow the electrode to be stimulated by a much higher-amplitude signal than possible by stimulation of the co-located AFE circuit 42b-2 alone. The four AFE circuits 42b-2, 42-2 may provide signals of different amplitudes to the electrode 100b-2, or signals of a same amplitude, or a combination (e.g., two signals having a same amplitude and two signals have different amplitudes from the other signals).

The electrode 100b-1 may be sensed concurrently by each of the two AFE circuits 42b-1, 42-1 to obtain two sensed signals from the electrode 100b-1, and the electrode 100b-2 may be sensed concurrently by each of the four AFE circuits 42b-2, 42-2 to obtain four sensed signals from the electrode 100b-2, according to some embodiments of the present technology. The two sensed signals may be provided to the back-end circuitry 80 and the host computer 90 for recording and/or further processing, or may be processed by on-chip circuitry that is part of the front-end circuitry 40 of the apparatus 1000. In some embodiments, the sensed signals may be processed, by the host computer 90 or by other on-chip or off-chip circuitry, to produce a combined signal having a higher SNR than the SNR of any individual one of the sensed signals. More specifically, if it is assumed that intrinsic noise in each of the AFE circuits 42b-1, 42b-2, 42-1, 42-2 is random, i.e., not correlated with each other, then the sensed signals may be averaged to reduce the random intrinsic noise from the AFE circuits 42b-1, 42b-2, 42-1, 42-2, which in effect may increase the overall SNR of the combined (i.e., averaged) signal. In some embodiments, the overall SNR of the combined signal may increase by the square root of the number N of sensed signals processed to obtain the combined signal, i.e., by \sqrt{N}. Therefore, for the case of the electrode 100b-1 connected to the two AFE circuits 42b-1, 42-1, the number of sensed signals is two (i.e., N=2) and the combined signal obtained by averaging the two sensed signals may have an overall SNR that is increased by approximately 40% or a factor of 1.4 compared with the SNR of any individual one of the sensed signals, according to \sqrt{2}≈1.4. Similarly, for the case of the electrode 100b-2 connected to the four AFE circuits 42b-2, 42-2, the number of sensed signals is four (i.e., N=4) and the combined signal obtained by averaging the four sensed signals may have an overall SNR that is double or increased by a factor of 2 compared with the SNR of any individual one of the sensed signals, according to \sqrt{4}=2.

As will be appreciated, the increase in the overall SNR of the combined signal by a factor of \sqrt{N}, where N is the number of sensed signals processed to obtain the combined signal, assumes that the noise at each of the AFE circuits is random while desired signal at each of the AFE circuits is the same. The randomness of the noise results in some of the noise cancelling out during the averaging process while the desired signal remains the same, thus reducing the noise portion of the SNR.

FIG. 2D schematically shows arrangements of first and second electrodes 100-1, 100-2, which may be densely arranged on the chip 10 relative to each other and/or relative to other electrodes 100 on the chip 10, according to some embodiments of the present technology. In some implementations, the first and second electrodes 100-1, 100-2 may be part of an array of densely arranged electrodes. The first and second electrodes 100-1, 100-2 are not co-located with any of the AFE circuits 42. The first electrode 100-1 may be connected to a pair of AFE circuits 42-1, which are located separately from the first electrode 100-1, via a routing bus 62a-1 and routing lines 62. The second electrode 100-2 may be connected to four AFE circuits 42-2, which are located separately from the second electrode 100-2, via a routing bus 62a-2 and routing lines 62. In FIG. 2D, switches 66-1 in the routing lines 62 connecting the electrode 100-1 to the AFE circuits 42-1 are in the closed state. The electrode 100-1 may therefore be stimulated concurrently by stimulation signals provided by the two AFE circuits 42-1 or may be sensed concurrently by the two AFE circuits 42-1, as with the electrode 100b-1 of FIG. 2C. Similarly, the electrode 100-2 may be stimulated concurrently by stimulation signals provide by the four AFE circuits 42-2 or may be sensed concurrently by the four AFE circuits 42-2, as with the electrode 100b-2 of FIG. 2C.

In some embodiments of the present technology, the electrodes 100b-1, 100b-2 of FIG. 2C may be sparse electrodes with sufficient distance between each other and other electrodes 100 of the apparatus 1000 that the electrodes 100b-1, 100b-2 may be electrically connected to an external device (e.g., a PCB, another chip, etc.) via wire bonding techniques or via flip-chip bonding techniques, for example, without interacting with the co-located AFE circuits 42b-1, 42b-2. In some embodiments, upper surfaces of the electrodes 100b-1, 100b-2 may be electrically connected to an external device while internal portions of the electrodes 100b-1, 100b-2 may be electrically connected to their respective AFE circuits 42b-1, 42b-2. The ability to have an external device communicate directly with the electrodes 100b-1, 100b-2 (i.e., not via the AFE circuits 42b-1, 42b-2) may be advantageous for direct stimulation of the electrodes 100b-1, 100b-2 via a signal from the external device instead of or in addition to the stimulation signals provided by the AFE circuits 42b-1, 42b-2, and/or for direct sensing of the electrodes 100b-1, 100b-2 by the external device instead of or in addition to the sensing performed by the AFE circuits 42b-1, 42b-2.

In some embodiments of the present technology, the electrodes 100-1, 100-2 of FIG. 2D may be part of an array of densely arranged electrodes 100 of the apparatus 1000 and may be able to, e.g., stimulate closely spaced features of a test subject and/or sense signals from closely spaced regions of the test subject.

The arrangements schematically depicted in FIGS. 2B, 2C, and 2D, as well as similar arrangements, which utilize the switching matrix 60 or a similar switching system, may be used advantageously to configure groups of one or more electrodes 100 to be connected individually to one, or two, or three, or more, AFE circuits 42 selectively and controllably, which may permit each of the connected electrodes 100 to be concurrently stimulated by a combined stimulation signal that is the sum of the signal(s) from the connected AFE circuits 42, and which may permit each of the connected electrodes 100 to be concurrently sensed by each of the connected AFE circuits 42. In one example, a first electrode 100 of the group may be stimulated by a first combined stimulation signal from a first group of AFE circuits 42, a second electrode 100 of the group may be stimulated by a second combined stimulation signal from second group of AFE circuits 42, a third electrode 100 of the group may be stimulated by a third combined stimulation signal from third group of AFE circuits 42, and so on. The first, second, and third groups of AFE circuits 42 may be different from each other or may be one and the same group of AFE circuits 42. Thus, as discussed herein, the switching matrix 60 or a similar switching system may be used advantageously to expand the range of amplitudes of stimulation signals provided to an electrode 100 of the apparatus 1000. In another example, the first electrode 100 of the group may be sensed by each AFE circuit 42 of the first group of AFE circuits 42, the second electrode 100 of the group may be sensed by each AFE circuit 42 of the second group of AFE circuits 42, the third electrode 100 of the group may be sensed by each AFE circuit 42 of the third group of AFE circuits 42, and so on. The first, second, and third groups of AFE circuits 42 may be different, such that sensing of the first, second, and third electrodes 100 may occur concurrently by the different groups of AFE circuits 42. Signals sensed by each group of AFE circuits may be averaged to yield a combined signal that has a higher SNR than any individual one of the sensed signals, as discussed herein. On the other hand, if sensing of the first, second, and third electrodes 100 is to occur at different times (e.g., sequentially), the first, second, and third groups of AFE circuits 42 may be one and the same group of AFE circuits 42. Thus, as discussed herein, the switching matrix 60 or a similar switching system may be used advantageously to increase the number of AFE circuits 42 able to sense signals currently from an electrode 100, which can yield a combined signal that has a SNR that increases with an increase in the number of connected AFE circuits 42, as discussed herein.

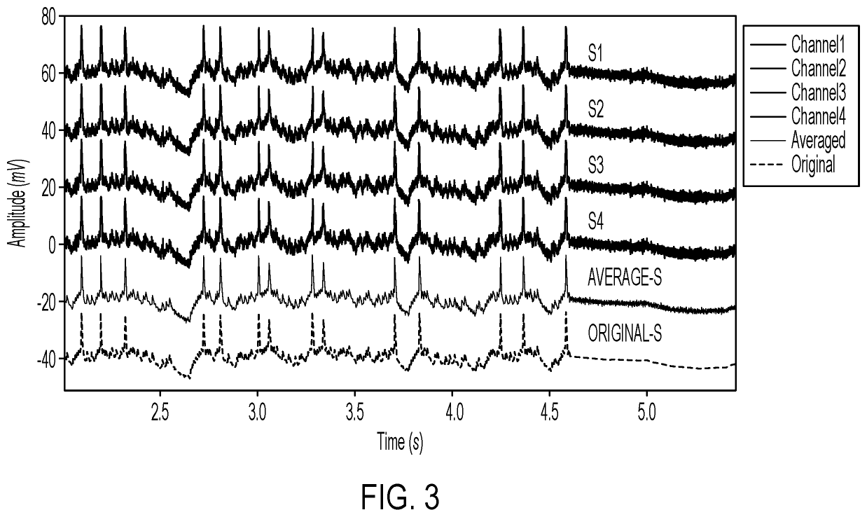

FIG. 3 shows traces of signal amplitude (in units of mV) as a function of time (in units of s) recorded for four signals S1, S2, S3, S4 sensed from an electrode by four different AFE circuits based on a test signal ORIGINAL-S applied to the electrode. For comparison with the four signals S1, S2, S3, S4, also shown in FIG. 3 are a trace for the test signal ORIGINAL-S and a trace for an averaged signal AVERAGE-S obtained by averaging the four signals S1, S2, S3, S4. The traces are an example of how the SNR may be increased by sensing an electrode (e.g., the electrode 100b-2 of FIG. 2B) using a plurality of AFE circuits (e.g., the four AFE circuits 42b-2, 42-2 of FIG. 2B) and combining the signals sensed by the plurality of AFE circuits by averaging the signals. As can be seen in FIG. 3, the traces for the four signals S1, S2, S3, S4 each have noticeable jitter or noise relative to the test signal ORIGINAL-S, which is believed to result from intrinsic noise in the AFE circuits. The intrinsic noise is not correlated between the AFE circuits, and the jitter in the signals S1, S2, S3, S4 appears to be different from each other. The four signals S1, S2, S3, S4 were combined and processed to obtain the averaged signal AVERAGE-S. As shown in FIG. 3, the noise in the averaged signal AVERAGE-S is considerably lower than in the signals S1, S2, S3, S4 sensed individually, and the averaged signal AVERAGE-S is much closer in appearance to the test signal ORIGINAL-S than any of the signals S1, S2, S3, S4.

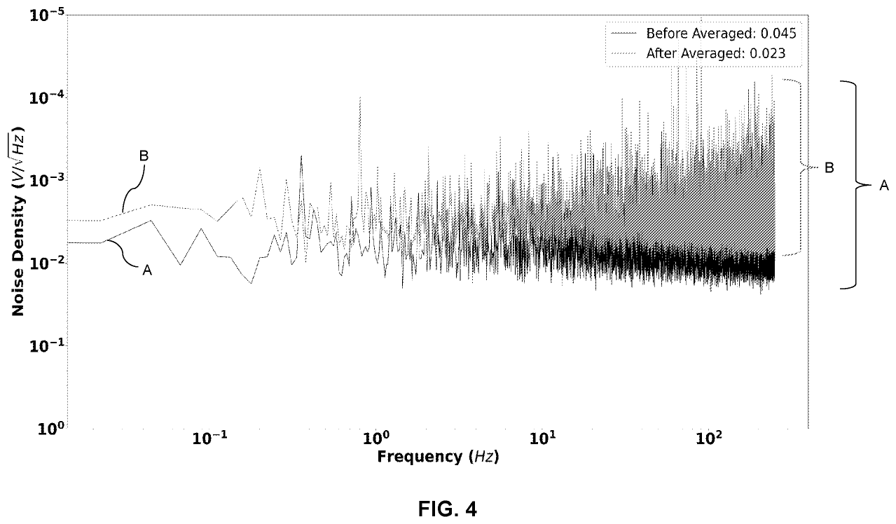

FIG. 4 shows traces of noise density (in units of V\sqrt{Hz}) as a function of frequency for a signal sensed from an electrode by a single AFE circuit (trace A) and an averaged signal computed from signals sensed from the electrode by four AFE circuits (trace B). Trace A was found to have relatively higher noise values compared with trace B for the entire range of frequencies observed. Also, for frequencies of 1 Hz and above, in the range of frequencies observed, trace A was found to have a larger scatter in noise values relative to trace B. A reduction in noise of approximately 50% was estimated for trace B relative to trace A.

The traces in FIGS. 3 and 4 demonstrate the ability to increase the SNR of a signal sensed from an electrode by increasing the number of AFE circuits connected to the electrode an able to sense signals from the electrode. By concurrently sensing and recording signals from a plurality of AFE circuits and averaging the signals to obtain an averaged signal, intrinsic noise in the AFE circuits may be reduced by a factor of \sqrt{N}, where N is the number of signals averaged to obtain the averaged signal and corresponds to the number of AFE circuits concurrently sensing signals from the electrode.

According to some embodiments of the present technology, an arrangement in which a plurality of electrodes (e.g., the array 20 of electrodes 100) is interconnected with a plurality of AFE circuits (e.g., the AFE circuits 42) via a switching system (e.g., the switching matrix 60) may be operated advantageously to increase the SNR of a signal sensed from an electrode after it is determined that it is difficult to differentiate the signal from noise sensed together with the signal by a single AFE circuit. The arrangement may be reconfigured such that multiple signals may be sensed from the electrode by setting the opened/closed states of switches in the arrangement to connect the electrode to n AFE circuits instead of the single AFE circuit, where n is a number greater than one. The n signals sensed by the n AFE circuits may be recorded and processed to obtain an averaged signal (n-averaged signal) having a higher SNR than the signal sensed by the single AFE circuit, as discussed above. If noise in the n-averaged signal is still unacceptably high, the arrangement may be further reconfigured by setting the opened/closed states of the switches to connect the electrode to m AFE circuits, where m is a number greater than n, and then sensing, recording, and processing m signals to obtain an m-averaged signal having a higher SNR than the n-averaged signal. This may continue until an averaged signal is obtained that has an acceptable amount of noise, such as when the signal sensed from the electrode is discernible from noise present in the sensed signal, i.e., the signal sensed from the electrode is not masked by the intrinsic noise from the AFE circuit(s). As will be appreciated, although various embodiments may be described for sensing one or more signal(s) from one electrode, the techniques and arrangements for sensing one electrode may be used to sense signals from multiple electrodes. For example, for a chip comprised of one thousand electrodes and twenty AFE circuits, the twenty AFE circuits may be selectively configured, via the opened/closed states of switches on the chip, to sense twenty of the electrodes concurrently, using a single AFE circuit per electrode, and then sensing another twenty of the electrodes concurrently, and so on, until all the electrodes are sensed. If the SNR is not acceptable for the sensed signals, the switches on the chip may be reconfigured to sense ten of the electrodes concurrently using two of the twenty AFE circuits per electrode, and then sensing another ten of the electrodes concurrently, and so on. If the SNR is still unacceptable, the switches on the chip may be reconfigured again to sense five of the electrodes concurrently using four of the twenty AFE circuits per electrode. In some embodiments, the switches on the chip may be configured such that one or more electrodes may be sensed by x AFE circuits per electrode while one or more other electrodes may be sensed by y AFE circuits, where x and y are different numbers.

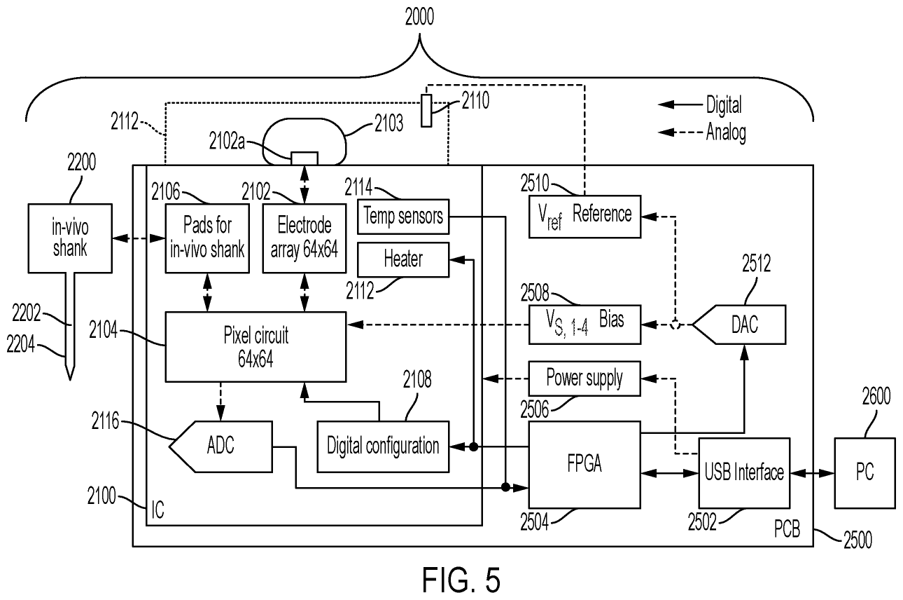

FIG. 5 schematically shows a multi-electrode apparatus 2000, according to some embodiments of the present technology. The apparatus 2000 may comprise an in vivo shank 2200 configured to be implanted in a live subject. For example, the shank 2200 may be a neurological probe that may be implanted in or near a region of the live subject's brain. The apparatus 2000 may comprise an integrated circuit (“IC”) 2100 in communication with electronic components on the shank 2200, and may comprise a PCB 2500 in communication with the IC 2100. In some embodiments, the IC 2100 may include some of or all of the components of the chip 10 discussed above. In some embodiments, the PCB 2500 may include some of or all of the components of the back-end circuitry 80 discussed above. The apparatus 2000 may be controlled by a computer 2600. In some embodiments, the computer 2600 may be in communication with the PCB 2500 via an input/output (“I/O”) interface 2502 of the PCB 2500. For example, the I/O interface 2502 may be a USB interface, a FireWire connector, a Thunderbolt interface, or the like. In some embodiments, the computer 2600 may include some of or all of the components of the host computer 90 discussed above, and also may be operably connected to a storage device (not shown). In some embodiments, the computer 2600 may be configured to transmit power and digital signals to the I/O interface 2502, and may be configured to receive digital signals from the I/O interface 2502.

According to some embodiments of the present technology, the apparatus 2000 may include a plurality of electrodes 2102, 2202 in communication with a plurality of front-end circuits 2104. In some embodiments, the electrodes 2102, 2102 may include an array of electrodes 2102 located on the IC 2100 and/or an array of electrodes 2202 located on a stem portion 2204 of the shank 2200. In some embodiments, some of or all of the front-end circuits 2104 may be co-located with the electrodes 2102 on the IC 2100. For example, the front-end circuits 2104 may comprise a 64×64 array of AFE pixel circuits, and the electrodes 2102 may comprise a 64×64 array of pixel electrodes disposed above the AFE pixel circuits to form a 64×64 array of pixels each including a pixel electrode and a pixel circuit. The electrodes 2102, 2202 may be connected to the front-end circuits 2104 via a switching matrix disposed at least partially on the IC 2100. In some embodiments, the switching matrix may be similar to the switching matrix 60 discussed above, aspects of which are depicted in FIGS. 2A through 2D. For example, a switching matrix (not shown) may interconnect some of or all of the front-end circuits 2104 to selected electrodes 2102a of the electrodes 2102 of the apparatus 2000, so that the selected electrodes 2102a may be addressed by the front-end circuits 2104 for sensing or for stimulation. In FIG. 5, a single selected electrode 2102a is arranged to stimulate and/or to sense a signal from a target 2103; however, it should be understood that other selected electrodes 2102a (not shown) may be arranged for stimulating and/or for sensing signals from other targets 2103 (not shown). In some embodiments, the targets 2103 may correspond to discrete objects. In some embodiments, the targets 2013 may correspond to different areas of a single object. Similarly, although not depicted in FIG. 5, the switching matrix (not shown) may interconnect some of or all of the front-end circuits 2104 to selected shank electrodes of the electrodes 2202 located on the stem 2204 of the shank 2200, so that the selected shank electrodes may be addressed by the front-end circuits 2104. In some embodiments, the electrodes 2202 disposed on the stem 2204 of the shank 2200 may be connected to the front-end circuits 2104 via wiring (not shown) connecting the electrodes 2202 to contact pads 2106 disposed on the IC 2100. The electrodes 2202 may be connected directly to the wiring or via electrically conductive traces and contact pads on the shank 2200.

As noted above, back-end circuitry of the apparatus 2000 may be disposed on the PCB 2500. According to some embodiments of the present technology, the back-end circuitry may include any one of or any combination of: a FPGA 2504, a power supply 2506, a bias-voltage source 2508, a reference-voltage source 2510, and a DAC 2512. In some embodiments, the PCB 2500 may receive control signals and power from the computer 2600 via the I/O interface 2502. The control signals may include digital signals relayed to the FPGA 2504. In some embodiments, the FPGA 2504 may output digital configuration signals to a configuration memory 2108 disposed on the IC 2010, based on the control signals from the computer 2600. The configuration memory 2108 may store the digital configuration signals as configuration bits for configuring one or more of the front-end circuits 2104. In some embodiments, the FPGA 2504 may output signals to the bias-voltage source 2508 and the reference-voltage source 2510 via the DAC 25012. The bias-voltage source 2508 may set a bias voltage based on a signal from FPGA 2504 and may output the bias voltage to the front-end circuits 2104 on the IC 2100. The reference-voltage source 2510 may set a reference voltage based on a signal from the FPGA 2504 and may output the reference voltage for use on the IC 2100 and/or near the IC 2100. For example, the reference voltage may be provided to a reference electrode 2110 configured to set a reference potential in a fluid environment 2112 of the electrodes 2102 and/or the electrodes 2202. In some embodiments, the IC 2100 may include a heater 2112 controlled by signals from the FPGA 2504, and also may include one or more temperature sensor(s) 2114 each configured to sense a local temperature at the IC 2100 and to output the sensed temperature to the FPGA 2504. The FPGA 2504 may be configured to adjust the signals transmitted to the heater 2112 based on the sensed temperature(s) received from the temperature sensor(s) 2114. In some embodiments, the power supply 2506 may receive power from the computer 2600 via the I/O interface 2502 and may provide power to various components of the IC 2100. In some embodiments, signals sensed by the electrodes 2102, 2202 may be transmitted to the FPGA 2504 via the front-end circuits 2104 and an ADC 2116 or may be processed by circuitry (not shown) on the IC 2100 before being transmitted to the FPGA 2504 via the ADC 2116. In turn, the FPGA 2504 may transmit the sensed signals or the processed signals to the computer 2600 via the I/O interface 2502.

According to some embodiments of the present technology, processing for obtaining an n-averaged signal calculated from n sensed signals from an electrode 2102, 2202, which has a higher SNR than an individual signal used to obtain the n-averaged signal, may be performed on the IC 2100 and/or on the PCB 2500 (e.g., by circuitry in the FPGA 2504) and/or by the computer 2600. In some embodiments, processing for increasing the SNR may include determining that the n-averaged signal is excessively masked by noise, reconfiguring the switches to increase the number of the front-end circuits 2104 connected to the electrode 2102, 2202 to m (m being greater than n), obtaining m concurrently sensed signals from the electrode 2102, 2202, and calculating an m-averaged signal from the m sensed signals. Additional iterations may be performed to further increase the SNR until an averaged signal is obtained having a sufficiently high SNR that it is discernible from noise.

In some embodiments, a signal sensed from a selected electrode 2102a may be an OCV between the selected electrode 2102a and the reference electrode 2110. A signal may be sensed by each of the concurrently connected front-end circuits 2104 connected to the electrode 2012a, as discussed above, and may be processed to obtain an averaged OCV having a higher SNR than the OCV sensed from a single one of the connected front-end circuits 2104.

According to some embodiments of the present technology, some components of the IC 2100 may operate using digital signals or analog signals or a combination of digital signals and analog signals. Similarly, in some embodiments, some components of the PCB 2500 may operate using digital signals or analog signals or a combination of digital signals and analog signals. In one example, depicted in FIG. 5, transmissions of digital signals are represented by the darker (black) arrows and transmissions of analog signals are represented by the lighter (red) arrows.

According to some embodiments of the present technology, the IC 2100 may be supported by the PCB 2500. For example, the IC 2100 may be mounted on the PCB 2500 and may be electrically connected to components on the PCB 2500 via bond wires.

According to some embodiments of the present technology, turning back to FIG. 2A, the maximum number of signals that may be averaged to increase the SNR of a signal sensed from an electrode 100 may be limited by the maximum number channels configured to transmit the sensed signals to on-chip buffers (not shown) of the front-end circuitry 40 or to transmit the sensed signals off-chip, for recording and processing outside of the chip 10. In some embodiments, each of the buses 64 may serve as a channel to an on-chip buffer or to an off-chip device. In some embodiments, the number of buses 64 may equal the number of AFE circuits 42, in which case all of the AFE circuits may be used concurrently to sense signals from one or more of the electrodes 100. In some embodiments, the number of buses 64 may be less than the number of AFE circuits 42, in which case the switches 66 may be controlled to permit the buses 64 to be selectively connected to a subset of the AFE circuits 42 on the chip 10 for transmitting sensed signals from the subset of the AFE circuits 42 to on-chip buffers and/or to off-chip devices. On the other hand, for stimulation purposes, each bus 42 may be connected concurrently to a plurality of AFE circuits 42 such that an external signal may be provided to the connected AFE circuits 42 concurrently via the bus 42.

Having thus described several aspects and embodiments of the present technology, it is to be appreciated that various alterations, modifications, and improvements will readily occur to those skilled in the art. Such alterations, modifications, and improvements are intended to be part of this disclosure, and are intended to be within the spirit and scope of the disclosure. Further, though advantages of the present disclosure are indicated, it should be appreciated that not every embodiment of the technology described herein will include every described advantage. Some embodiments may not implement any feature(s) described as advantageous herein and in some instances one or more of the described features may be implemented to achieve further embodiments. Accordingly, the foregoing description and drawings are by way of example only.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in connection with one embodiment may be combined in any manner with aspects described in connection with one or more other embodiments.

Also, the present disclosure may be embodied as one or more method(s) in which various embodiments of the structures described above may be used. The acts performed as part of the one or more method(s) may be ordered in any suitable way.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

What is claimed is:

1. A multi-electrode apparatus, comprising:
a plurality of electrodes;
a plurality of circuits configured to provide signals to and/or sense signals from the electrodes;
routing lines comprising switches, the routing lines being configured to selectively connect the electrodes to the circuits; and
a control circuitry configured to control the switches such that at least one of the electrodes is connected concurrently to two or more of the circuits.

2. The apparatus of claim 1, wherein the control circuitry is configured to control the switches such that the two or more of the circuits are connected concurrently to a single one of the electrodes.

3. The apparatus of claim 1 or claim 2, wherein the control circuitry is configured to control the switches such that the two or more of the circuits are connected concurrently to at least two of the electrodes.

4. The apparatus of any one of claims 1 through 3, wherein the control circuitry is configured to control the switches such that:
a first group of one or more of the electrodes is connected concurrently to a first plurality of the circuits, and
a second group of one or more one of the electrodes is connected concurrently to a second plurality of the circuits.

5. The apparatus of any one of claims 1 through 4, wherein the control circuitry is configured to receive configuration data from an external device and to store the configuration data in the configuration memory as configuration bits for configuring a state of each of the switches.

6. The apparatus of any one of claims 1 through 5, wherein the circuits are configured to perform any one or any combination of:
sense a current,
sense a voltage,
output a stimulation current, and
output a stimulation voltage.

7. The apparatus of any one of claims 1 through 6, wherein the circuits are analog circuits.

8. The apparatus of any one of claims 1 through 7, wherein the circuits and at least some of the electrodes are disposed on a single substrate.

9. The apparatus of any one of claims 1 through 8, wherein the single substrate is a semiconductor chip.

10. The apparatus of any one of claims 1 through 9, wherein the electrodes comprise at least one electrode array.

11. The apparatus of any one of claims 1 through 10, wherein the at least one electrode array includes a first array of at least two electrodes disposed on the semiconductor chip.

12. The apparatus of any one of claims 1 through 11, further comprising:
a shank configured to be inserted in an object,
wherein the at least one electrode array includes:
a first array of at least two electrodes disposed on the semiconductor chip, and
a second array of at least two electrodes disposed on the shank.

13. The apparatus of any one of claims 1 through 12, wherein a total number of the circuits on the semiconductor chip is less than a total number of the electrodes.

14. The apparatus of any one of claims 1 through 13, wherein the control circuitry is configured to set the switches in a first configuration in which a selected group of electrodes is connected to the circuits such that each electrode of the selected group is connected to at least two of the circuits.

15. The apparatus of any one of claims 1 through 14, wherein the control circuitry is configured to set the switches in a second configuration such that each electrode of the selected group is connected to at least four of the circuits.

16. The apparatus of any one of claims 1 through 15, wherein the control circuitry is configured to set the switches in the second configuration based on whether a signal-to-noise (SNR) ratio of a first averaged signal determined from signals sensed by the at least wo of the circuits is at or above a threshold.

17. The apparatus of any one of claims 1 through 16, wherein, for each of the at least one of the electrodes:
the two or more of the circuits connected concurrently to the electrode are each configured to sense a signal from the electrode and to provide an output signal to the controller, and
the control circuitry is configured to:
receive the output signals from the two or more of the circuits,
compute an average of the output signals, and
record the average as a reading for the electrode.

18. The apparatus of any one of claims 1 through 17, wherein, for each of the at least one of the electrodes, the two or more of the circuits connected concurrently to the electrode are each configured to provide a stimulation signal to the electrode concurrently.

19. The apparatus of any one of claims 1 through 18, wherein the stimulation signals provided by the two or more of the circuits have a same amplitude.

20. The apparatus of any one of claims 1 through 19, wherein the stimulation signals provided by the two or more of the circuits have different amplitudes.

21. The apparatus of any one of claims 1 through 20, wherein, for each of the at least one of the electrodes, the two or more of the circuits connected concurrently to the electrode are each configured to provide a stimulation signal to the electrode concurrently and to provide the stimulation signal to another electrode concurrently.

22. The apparatus of any one of claims 1 through 21, further comprising:
bus lines configured to connect the circuits to an off-chip device, wherein:
a total number of the bus lines is equal to or greater than a total number of the circuits, and
each of the circuits is connected to at least one of the bus lines.

23. The apparatus of any one of claims 1 through 22, further comprising:
a circuit board on which the chip is mounted, the circuit board comprising back-end circuitry,
wherein:
the circuits are front-end circuits, and
the bus lines connect the front-end circuits to the back-end circuitry.

24. The apparatus of any one of claims 1 through 23, wherein:
the back-end circuitry includes a field-programmable gate array (FPGA), and
the bus lines connect the front-end circuits to the FPGA.

25. The apparatus of any one of claims 1 through 24, wherein the back-end circuitry includes any one or any combination of:
a power supply,
an input/output (I/O) interface,
a bias-voltage source, and
a reference-voltage source.

26. The apparatus of any one of claims 1 through 25, wherein the electrodes are part of a microelectrode array (MEA) of electrodes and are configured to perform in vitro sensing and/or stimulation of an object.

27. The apparatus of any one of claims 1 through 26, wherein the in vitro sensing and/or stimulation is extracellular.

28. The apparatus of any one of claims 1 through 27, wherein the in vitro sensing and/or stimulation is intracellular.

29. The apparatus of any one of claims 1 through 28, wherein the electrodes are part of a microelectrode array (MEA) of electrodes and are configured to perform in vivo sensing and/or stimulation of an object.

30. The apparatus of any one of claims 1 through 29, wherein the in vivo sensing and/or stimulation is extracellular.

31. The apparatus of any one of claims 1 through 30, wherein the in vivo sensing and/or stimulation is intracellular.

32. The apparatus of any one of claims 1 through 31, wherein the electrodes are configured to perform local field potential (LFP) sensing of the object.

33. The apparatus of any one of claims 1 through 32, wherein the electrodes are configured to perform open-circuit voltage (OCV) sensing of the object.

34. The apparatus of any one of claims 1 through 33, wherein:
at least some of the electrodes are disposed in a first area of the chip, and
at least some of the circuits are disposed in second area of the chip different from the first area.

35. The apparatus of any one of claims 1 through 34, wherein the first area is at least partially surrounded by the second area.

36. The apparatus of any one of claims 1 through 35, wherein:
at least some of the electrodes are disposed in a first area of the chip,
at least some of the circuits are disposed in the first area of the chip, and
in the first area of the chip, the electrodes are located above or below the circuits in a stacked arrangement.

37. The apparatus of any one of claims 1 through 36, wherein some of the electrodes are disposed in an area of the chip different from the first area.

38. The apparatus of any one of claims 1 through 37, wherein some of the circuits are disposed in an area of the chip different from the first area.

39. A method of using a multi-electrode apparatus, comprising:
controlling a plurality of switches in routing lines to selectively connect a plurality of electrodes to a plurality of circuits configured to provide signals to and/or sense signals from the electrodes,
wherein the controlling controls the switches such that at least one of the electrodes is connected concurrently to two or more of the circuits.

40. The method of claim 39, wherein the controlling controls the switches such that the two or more circuits are connected concurrently to a single one of the electrodes.

41. The method of claim 39 or claim 40, wherein the controlling controls the switches such that the two or more circuits are connected concurrently to at least two of the electrodes.

42. The method of any one of claims 39 through 41, wherein the controlling controls the switches such that:
a first group of one or more of the electrodes is connected concurrently to a first plurality of the circuits, and
a second group of one or more one of the electrodes is connected concurrently to a second plurality of the circuits.

43. The method of any one of claims 39 through 42, further comprising:
receiving, from an external device, configuration data for configuring a state of each of the switches; and
storing the configuration data in the configuration memory as configuration bits.

44. The method of any one of claims 39 through 43, wherein the circuits are configured to perform any one or any combination of:
sense a current,
sense a voltage,
output a stimulation current, and
output a stimulation voltage.

45. The method of any one of claims 39 through 44, wherein the circuits are analog circuits.

46. The method of any one of claims 39 through 45, wherein the electrodes and the circuits are disposed on a single substrate.

47. The method of any one of claims 39 through 46, wherein the single substrate is a single semiconductor chip.

48. The method of any one of claims 39 through 47, wherein the electrodes comprise at least one electrode array.

49. The method of any one of claims 39 through 48, wherein a total number of the circuits on the semiconductor chip is less than a total number of the electrodes on the semiconductor chip.

50. The method of any one of claims 39 through 49, wherein the controlling sets the switches in a first configuration in which a selected group of electrodes is connected to the circuits such that each electrode of the selected group is connected to a first number of the circuits.

51. The method of any one of claims 39 through 50, wherein, based on whether a signal-to-noise (SNR) ratio of a first signal determined from signals sensed by the first number of the circuits is at or above a threshold, the controlling sets the switches in a second configuration in which the selected group of electrodes is connected to the circuits such that each electrode of the selected group is connected to a second number of the circuits, the second number being greater than the first number.

52. The method of any one of claims 39 through 51, wherein:
the first number of the circuits is at least one of the circuits, and
the second number of the circuits is at least four of the circuits.

53. The method of any one of claims 39 through 52, further comprising, for each of the at least one of the electrodes:
sensing, by the two or more of the circuits connected concurrently to the electrode, a signal from the electrode; and
computing an average of the signals sensed by the two or more of the circuits and recording the average as a reading for the electrode.

54. The method of any one of claims 39 through 53, wherein the signals sensed by the two of more of the circuits are local field potentials (LFPs).

55. The method of any one of claims 39 through 54, wherein the signals sensed by the two of more of the circuits are open-circuit voltages (OCVs).

56. The method of any one of claims 39 through 55, further comprising, for each of the at least one of the electrodes:
stimulating the electrode with two or more stimulation signals respectively provided by the two or more of the circuits connected concurrently to the electrode.

57. The method of any one of claims 39 through 56, wherein the two or more stimulation signals are stimulation currents.

58. The method of any one of claims 39 through 57, wherein the two or more stimulation signals provided by the two or more of the circuits have a same amplitude.

59. The method of any one of claims 39 through 58, wherein the two or more stimulation signals provided by the two or more of the circuits have different amplitudes.

60. The method of any one of claims 39 through 59, wherein the two or more stimulation signals provided by the two or more of the circuits are provided to at least two of the electrodes concurrently.
